(19) United States
(12) Patent Application Publication
(10) Pub. No.: US 2007/0021591 A1
(43) Pub. Date: Jan. 25, 2007

(54) COMPOSITIONS AND METHODS FOR PROTEIN DEAGGREGATION

(75) Inventors: Arthur James Movius IV, Seattle, WA (US); Rajesh Dua, Issaquah, WA (US)

Correspondence Address: SPECKMAN LAW GROUP PLLC, 1201 THIRD AVENUE, SUITE 330, SEATTLE, WA, 98101, US

(73) Assignee: TRUBION PHARMACEUTICALS, INC., Seattle, WA (US)

(21) Appl. No.: 11/459,544

(22) Filed: Jul. 24, 2006

Related U.S. Application Data
(60) Provisional application No. 60/702,545, filed on Jul. 25, 2005.

(51) Int. Cl. C07K 14/705; C07K 16/18

(52) U.S. Cl. 530/350; 530/388.1

(57) ABSTRACT

Compositions and methods are provided for achieving the deaggregation of binding proteins including, but not limited to, protein ligands, soluble receptors, antibodies, antibody fragments, variable fragment single-chain antibodies (scFv), and small modular immunopharmaceutical products (SMIP™ products). The compositions, which are suitable for the deaggregation of highly concentrated solutions of binding proteins, contain one or more chaotrope, are typically formulated at an acidic pH, and may be used to provide binding proteins suitable for the preparation of pharmaceutical compositions and administration in vivo to a patient.

### CROSS REFERENCE TO RELATED APPLICATIONS

This application claims priority under 35 U.S.C. § 119(e) to U.S. Provisional Application No. 60/702,545, filed Jul. 25, 2005.

### TECHNICAL FIELD OF THE INVENTION

The present invention relates to the fields of protein chemistry and recombinant DNA technology. More specifically, the present invention provides compositions and methods for achieving the deaggregation of binding proteins including, but not limited to, protein ligands, soluble receptors, antibodies, antibody fragments, variable fragment single-chain antibodies (scFv), and small modular immunopharmaceutical products (SMIP™ products).

### BACKGROUND OF THE INVENTION

Recombinant DNA methodologies permit the large-scale production of genetically engineered proteins. Such methodologies for producing recombinant proteins are well known in the art. Typically, a DNA segment encoding a particular protein is inserted into a host microorganism and the transformed microorganism is grown under conditions that induce heterologous protein expression.

Commonly, however, heterologous proteins expressed in bacteria, typically E. coli, are not biologically active because they do not fold into the proper tertiary structure but, rather, form large aggregates of inactive protein referred to as inclusion bodies. Inclusion bodies may also be caused by the formation of covalent intermolecular disulfide bonds that link together several protein molecules to form insoluble complexes. Steps must be taken to denature and refold these proteins to restore biological activity.

In addition to expression in microorganisms, there also exist methodologies for expressing recombinant proteins in eukaryotic cells, including yeast, insect cells, and a wide variety of mammalian cells. Regardless of the cells used for gene expression, however, the synthesized recombinant proteins must fold and assemble into a proper tertiary structure in order to have biological activity.

It is well understood that a family of proteins referred to as molecular chaperones are required to mediate the folding process. In the absence of the appropriate molecular chaperone, newly expressed recombinant proteins aggregate thereby preventing the formation of functional proteins. Goloubinoff et al., Nature 342:884-889 (1989) and Welch, Scientific American 56-64 (May 1993). Despite the existence of chaperones, aggregation of protein still occurs in vivo and may, in fact, contribute to, or cause, various disease states such as Down's syndrome, Alzheimer's disease, diabetes, and cataracts. De Young et al., Accounts of Chemical Research 26:614-620 (1993); Wetzel, TIBTECH 12:193-198 (1994); and Haass and Selkoe, Cell 75:1039-1042 (1993).

A wide range of recombinant proteins including enzymes and binding proteins, such as antibodies, antibody fragments, scFv, and SMIP™ products, are susceptible to loss of activity and/or to formation of soluble or insoluble aggregates, such as trimers and higher polymers, in aqueous solutions, when stored at low temperatures (i.e. below 0° C.), and when subjected to repeated cycles of freezing and thawing. Protein aggregation is of major importance to the biotechnology industry because of the importance of in vitro production of recombinant proteins. Proteins in solution, even highly purified proteins, can form aggregates upon storage, or during production processes. In vitro aggregation limits protein stability, solubility, and production yields of recombinant proteins. De Young et al., Accounts of Chemical Research 26:614-620 (1993); Wetzel, TIBTECH 12:193-198 (1994); and Vandenbroeck et al., Eur. J. Biochem. 215:481-486 (1993).

In addition to contributing to loss of biological activity, protein aggregation can be harmful in therapeutic uses. In some cases, aggregate formation leads to complexes having increased immunogenicity when administered in vivo. Thus, in order to administer a recombinant protein solution to a patient, it is necessary to first remove these aggregates to avoid a toxic response by the patient. Consequently, the formation of aggregates of recombinant proteins is unacceptable for the preparation of pharmaceutical compositions.

A number of methodologies and additives have been described in the art for stabilizing protein solutions thereby preventing and/or minimizing the formation of protein aggregates. Conventional filtration processes have been described; however, aggregates—even at concentrations as low as 0.1-0.2%—rapidly clog such filters limiting their utility for manufacturing processes. Gel filtration chromatography and size exclusion chromatography methodologies are often effective, but very expensive and, therefore, impractical.

The stabilization of proteins by addition of heat-shock proteins such as HSP25 is described in EP-A0599344. The use of block polymers composed of polyoxy-propylene and polyoxy-ethylene and phospholipids has been described for the stabilization of antibody solutions. EP-A0318081. The stabilization of immunoglobulins by addition of a salt of a basic substance containing nitrogen such as arginine, guanine, or imidazole is described in EP-A0025275. Other additives for stabilization have also been described, including polyethers (EP-A0018609); glycerin, albumin, and dextran sulfate (U.S. Pat. No. 4,808,705); detergents such as Tween®20 (DE 2652636 and GB 8514349); chaperones such as GroEL (Mendoza, Biotechnol. Tech. 10:535-540 (1991)) and B23 (U.S. Pat. No. 6,358,718); citrate buffer (WO 93/22335); and chelating agents (WO 91/15509).

Existing methodologies for achieving protein deaggregation commonly employ the step of solubilizing the protein in high concentrations of strong chaotropes, such as 8M guanidine hydrochloride and/or urea, or surfactant, which results in nearly complete protein unfolding. Mitraki et al., Eur. J. Biochem. 163:29-34 (1987); Vandenbroeck et al., Eur. J. Biochem. 215:481-486 (1993); DeLoskey et al., Arch. Biochem. Biophys. 311:72-78 (1994); and Rudolph and Lilie, FASEB J. 10:49-56 (1996). Once soluble and unfolded, the proteins are diluted in additional chaotrope and refolded by removing the chaotrope, for example, by dialysis. Such refolding of proteins is quite unpredictable and condition dependent. Valax and Georgiou, Biotech. Prog. 9:539-547 (1993). Redox conditions, pH, dialysis rates, and protein concentrations must be empirically optimized on a protein-by-protein basis. And, re-aggregation is generally favored over proper refolding. As a result, acceptable yields of refolded protein often require that the protein be refolded at very low concentrations (e.g., 10-100 μg/ml). Rudolph, Modern Methods in Protein and Nucleic Acid Research 149-172 (Tschesche ed., 1990); Goldberg et al., Biochem. 30:2790-2797 (1991); and Maachupalli-Reddy et al., Biotech. Prog. 13:144-150 (1997). Once refolded, the protein must, therefore, be concentrated by 100-1000 fold to achieve a suitable concentration for in vivo administration—a process that typically results in substantial loss of native protein. Furthermore, the large volumes required for protein refolding result in the waste of substantial quantities of expensive reagents, such as chaotropes.

U.S. Pat. No. 5,077,392 discloses a method for activating recombinant proteins produced in prokaryotic cells wherein the aggregated proteins are dissolved in 4-8M guanidine hydrochloride or 6-10M urea. The resulting protein solutions are dialyzed to a pH of between 1 and 4 before being subjected to a nondenaturing and oxidizing environment to permit protein refolding.

U.S. Pat. No. 5,593,865 discloses a method for activating recombinant disulfide bond-containing eukaryotic proteins expressed in prokaryotic host cells. Inclusion bodies are dissolved in 6M guanidine hydrochloride containing reducing agents. In a refolding step, proteins are introduced into an oxidizing and nondenaturing environment.

U.S. Pat. No. 4,659,568 discloses a method for solubilizing, purifying, and characterizing protein from insoluble protein aggregates or complexes. The insoluble protein aggregates are layered on a urea step gradient (3M to 7M urea). As the samples are centrifuged, the aggregates pass through the gradient until dissolved.

U.S. Pat. No. 5,728,804 discloses a method wherein denatured or aggregated proteins are suspending in a detergent-free aqueous medium containing 5-7M guanidine hydrochloride and subjected to overnight incubation. The suspended sample is contacted with cyclodextrin to promote protein refolding.

U.S. Pat. No. 4,652,630 discloses a method for producing active somatotropin by solubilizing aggregates or inclusion bodies in a chaotrope (3M to 5M urea). The pH is adjusted to achieve complete solubilization followed by modifying the conditions to permit oxidation in the presence of nondenaturing concentrations of chaotrope.

U.S. Pat. No. 5,064,943 discloses a method for solubilizing and renaturing somatotropin without the use of a chaotrope. By this method, the pH is adjusted to between 11.5 and 12.5 and maintained for 5 to 12 hours thereby achieving the solubilization and renaturation of somatotropin.

U.S. Pat. No. 5,023,323 discloses a method for deaggregating somatotropin aggregates wherein the aggregates are dissolved in a denaturing chaotrope (1M to 8M urea). Following solubilization, the sample is exposed to a nondenaturing, oxidizing environment.

U.S. Pat. No. 5,109,117 discloses a method for deaggregating somatotropin aggregates by dissolving in the presence of an organic alcohol and chaotrope (1M to 8M urea) followed by renaturing the solubilized protein in a nondenaturing, oxidizing environment.

U.S. Pat. No. 5,714,371 discloses a method for refolding aggregates of hepatitis C virus protease by solubilizing in 5M guanidine hydrochloride. A reducing agent is added to the solution and the pH adjusted to an acid pH. The denaturing agent is removed by dialysis and the pH raised.

U.S. Pat. No. 4,923,967 discloses a method for deaggregating human interleukin-2 (IL-2) wherein protein aggregates are dissolved in 4-8M guanidine hydrochloride with a sulfitolyzing agent. The sulfitolyzing agent is subsequently removed by solvent exchange and the temperature is raised to precipitate out pure IL-2. The protein is refolded by dissolving the precipitate in guanidine hydrochloride plus a reducing agent followed by dilution to permit protein refolding.

U.S. Pat. No. 5,410,026 discloses a method for refolding insoluble, misfolded insulin-like growth factor-1 (IGF-1) into an active conformation. Isolated protein is incubated with 1-3M urea or 1M guanidine hydrochloride until the aggregates are solubilized and refolded.

Variable fragment single-chain antibodies (scFv) are aggregation prone proteins having important diagnostic and therapeutic medical applications including tumor imaging and targeted drug delivery. Although complex expression systems have been developed that provide soluble and functional scFv, the yield and concentration obtained is often less than desired. Bacterial expression funnels large amounts of scFv into inclusion bodies, preventing the scFv from folding into an active form. Methods to recover functional scFv from inclusion bodies suffer drawbacks such as aggregate formation and require the use of large quantities of denaturants such as guanidine hydrochloride.

The structural similarities of scFv with proteins implicated in aggregation-driven human diseases and the need for a highly efficient, fast, inexpensive, and aggregate-free recovery method for scFv from bacterial systems warrant research into the aggregation behavior of these proteins. The inability of current chemical techniques to sufficiently hinder aggregation has focused attention on physical treatments that show promise at reversing aggregation. Prior studies have shown that aggregates of multimeric proteins break apart and subsequently regain activity following exposure to high pressure. High pressure treatments, in conjunction with current chemical methods, may provide one solution to the aggregation problem in scFv production.

Small modular immunopharmaceutical products (SMIP™ products) are a highly modular, antibody-based compound class having enhanced drug properties over monoclonal and recombinant antibodies. SMIP™ products comprise a single polypeptide chain including a target-specific binding domain, based, for example, upon an antibody variable domain, in combination with a variable FC region that permits the specific recruitment of a desired class of effector cells (such as, e.g., macrophages and natural killer (NK) cells) and/or recruitment of complement-mediated killing. Depending upon the choice of target and hinge regions, SMIP™ products can signal or block signalling via cell surface receptors.

Like scFv, SMIP™ products are highly susceptible to formation of protein aggregates upon in vitro expression in a heterologous host cell. Preliminary studies on deaggregation of SMIP™ products demonstrated that high concentrations of urea (e.g., 6M) at neutral pH (i.e. phosphate buffered saline pH 7.0) are effective in deaggregating SMIP™ products in solutions comprising low concentrations of protein (i.e. less than 1 mg/ml). Unfortunately, however, higher protein concentrations resulted in the accumulation of very high molecular weight aggregates and loss of total protein. Furthermore, it was found that the length of time of incubation with 6M urea was limited to 5 hours or less; extended incubation times resulted in the formation of very high molecular weight (HMW) aggregates.

There remains a substantial unmet need in the art for compositions and methods to achieve the deaggregation of binding proteins in high concentrations suitable for the preparation of pharmaceutical compositions and for the in vivo administration to patients.

### SUMMARY OF THE INVENTION

The present invention addresses these and other related needs by providing, inter alia, compositions and methods for recovering biologically active binding proteins from mixtures containing aggregates. Methods presented herein provide the deaggregation of aggregates present in mixtures of aggregated and deaggregated (i.e. native) protein. Compositions and methods presented herein are effective in achieving the deaggregation of binding proteins including, but not limited to, protein ligands, soluble receptors, antibodies, antibody fragments, variable fragment single-chain antibodies (scFv), and small modular immunopharmaceutical products (SMIP™ products).

Compositions and methods disclosed herein may be suitably employed with solutions of binding protein in the range of between about 0.1 mg/ml to about 50 mg/ml, more typically between about 1 mg/ml and about 50 mg/ml, still more typically between about 1 mg/ml and about 25 mg/ml or between about 1 mg/ml and about 10 mg/ml. Exemplified herein are compositions and methods for deaggregating binding proteins in solutions comprising about 1 mg/ml, about 2 mg/ml, about 5 mg/ml, about 8 mg/ml, or about 10 mg/ml total binding protein.

Compositions and methods disclosed herein generally comprise buffer systems that are compatible with GMP manufacturing processes. For example, suitable buffer systems may include one or more salt(s) including, but not limited to, sodium acetate (NaOAc) and/or sodium chloride (NaCl). Suitable concentration ranges for each of these salts is from about 1 mM to about 100 mM, more typically from about 5 mM to about 50 mM or from about 10 mM to about 25 mM. Exemplified herein is a buffer system comprising 25 mM NaOAc and 25 mM NaCl.

The compositions and methods for deaggregating binding proteins presented herein additionally comprise one or more chaotropic agent(s) including, but not limited to, one or more of guanidine hydrochloride, arginine, and urea. It will be understood that the precise concentration of chaotropic agent will depend upon the nature of the binding protein and its sensitivity to the chaotropic agent, but will be limited to concentrations that permit retention of biological activity of the protein in its native form. Typically, each chaotropic agent(s) is present in compositions at a concentration range from about 0.1M to about 8M. More typically, each chaotropic agent(s) is present at a concentration range from about 0.5M to about 6M, even more typically from about 1M to about 5M or from about 3M to about 5M. Exemplified herein are compositions comprising one or more chaotrope(s) at concentrations of about 3M, 3.5M, 4M, 4.5M and 5M.

Within related aspects, it will be appreciated that synergistic effects between combinations of two or more chaotropes may be advantageously achieved. For example, the present invention contemplates compositions and methods employing urea in combination with guanidine hydrochloride, urea in combination with arginine, and guanidine hydrochloride in combination with arginine. Regardless of the combination of chaotropes employed, each chaotropic agent(s) is present in compositions at a concentration range from about 0.1M to about 8M. More typically, each chaotropic agent(s) is present at a concentration range from about 0.5M to about 6M, even more typically from about 1M to about 5M or from about 3M to about 5M.

Regardless of the precise salt and chaotrope identity and concentration, compositions provided herein are typically adjusted to a slightly acidic pH, typically in the range from about pH 4 to about pH 7, more typically in the range from about pH 5 to about pH 6. Exemplified herein are compositions buffered to about pH 5, about pH 5.5, and about pH 6. It will be understood that, as a general rule, compositions comprising higher concentrations of chaotropic agent(s) are typically buffered to a higher pH whereas compositions comprising lower concentrations of chaotropic agent(s) are typically buffered to a lower pH. Thus, for example, compositions comprising a chaotropic agent at about 3M are typically buffered to about pH 5 whereas compositions comprising a chaotropic agent at about 4M are buffered to about pH 6. Other suitable compositions comprise a chaotropic agent at about 3.5M, which are buffered to about pH 5.5. Other buffer systems may be suitably employed.

Using buffer systems as described above, high levels of deaggregation are achieved with one or more chaotropes at concentrations of between about 3M and about 4M urea over a time period of up to about 5 hours to about 24 hours. As described in further detail herein, the activity of the binding protein is insensitive to protein concentration and the accumulation of high molecular weight (HMW) aggregates is substantially reduced.

Within certain aspects of the present invention, compositions and methods may additionally comprise one or more reducing agents such as, for example, Tris(2-carboxyethyl)phosphine hydrochloride (TCEP), beta-mercaptoethanol (BME), dithiothreitol (DTT), and glutathione (GSH). It will be appreciated by those of skill in the art that the addition of reducing agents is particularly advantageous for use with binding proteins wherein intra- and/or inter-molecular disulfide bonds are not required to provide stabilization of the protein's tertiary and/or quaternary structure. DTT is typically present in compositions at between about 1 mM and about 50 mM. GSH is typically present in compositions at between about 1 μM and about 100 μM, more typically between about 5 μM and about 20 Within still further aspects, compositions and methods may additionally or alternatively comprise one or more chelating agents exemplified by DTPA (Diethylenetriaminepentaacetic acid; Diethylenetriamine-N,N,N′,N′,N″-pentaacetic acid; Pentetic acid; N,N-Bis(2-(bis-(carboxymethyl)amino)ethyl)-glycine; Diethylenetriamine pentaacetic acid, [[(Carboxymethyl)imino]bis(ethylenenitrilo)]-tetra-acetic acid); EDTA (Edetic acid; Ethylenedinitrilotetraacetic acid; EDTA, free base; EDTA free acid; Ethylenediamine-N,N,N′,N′-tetraacetic acid; Hampene; Versene; N,N′-1,2-Ethane diylbis-(N-(carboxymethyl)glycine); Ethylene Diamine Tetraacetic Acid); and NTA (N,N-bis(carboxymethyl)glycine; Triglycollamic acid; Trilone A; alpha,alpha′,alpha″-trimethylaminetricarboxylic acid; Tri(carboxymethyl)amine; Aminotriacetic acid; Hampshire nta acid; nitrilo-2,2′,2″-triacetic acid; Titriplex i; Nitrilotriacetic acid). Other chelating agents may be suitably employed.

The deaggregation of a wide variety of binding proteins may be satisfactorily obtained with the compositions and methods presented herein. Exemplified are binding proteins having specific binding affinity for CD20, VEGF, Her2, EGFR, or CD37. For example, the present invention is exemplified by compositions and methods for deaggregation of a SMIP™ product having specific binding affinity for CD20.

Binding proteins deaggregated by the compositions and methods of the present invention display substantial levels of in vitro activity as evidenced by binding and functional assays as well as substantial levels of in vivo activity. For example, the CD20 specific SMIP™ product presented herein displays substantial levels of specific binding to CD20 antigen expressed on the surface of the WIL-2S cell line as well as substantial levels of complement-dependent cytotoxicity (CDC) activity in an in vitro complement fixation assay.

These and other aspects of the present invention will become apparent upon reference to the following detailed description. All references disclosed herein are hereby incorporated by reference in their entirety as if each was incorporated individually.

### BRIEF DESCRIPTION OF THE FIGURES

FIG. 1 presents a chromatographic trace showing the time-dependent elution of protein aggregates and POI for an exemplary CD20-specific SMIP™ product from a Protein A chromatography column eluted with a single step of Protein A at pH 5. The data presented in FIG. 1A were obtained with a binding protein applied to the column in a control buffer comprising 25 mM NaCl, 25 mM NaOAc at pH 5, whereas the data presented in FIG. 1B were obtained with the same binding protein applied to the column following a 20-hour treatment with a solution comprising 25 mM NaCl, 25 mM NaOAc, 3M urea at pH 5. The percentage of “protein of interest”, or % POI, obtained in FIG. 1A was 46.8% whereas the % POI obtained in FIG. 1B was 80.1%.

FIG. 2 is a bar graph demonstrating improved yields (expressed as % POI) for an exemplary CD20-specific SMIP™ product (5 mg/ml and 10 mg/ml) employing compositions and methods of the present invention (i.e. 25 mM NaOAc, 25 mM NaCl, 3M urea, pH 5 and 25 mM NaOAc, 25 mM NaCl, 4M urea, pH 5) in contrast to % POI for the same binding protein in phosphate buffered saline (PBS), pH 7 in combination with 3M urea or 4M urea.

FIG. 3 is a graph depicting the time-dependent concentration of POI (expressed as “area under curve” or AUC) for an exemplary CD20-specific SMIP™ product in the indicated compositions comprising 2M, 3M, or 4M urea each at pH 4, pH 5, and pH 6.

FIG. 4 presents a bar graph demonstrating improved yields (% POI, FIG. 4A and POI-AUC, FIG. 4B) for an exemplary CD20-specific SMIP™ product employing exemplary compositions and methods of the present invention (i.e. 25 mM NaOAc, 25 mM NaCl, 3M urea, pH 5 and 25 mM NaOAc, 25 mM NaCl, 4M urea, pH 6).

### DETAILED DESCRIPTION OF THE INVENTION

As indicated above, the present invention is directed to compositions and methods for the deaggregation of binding proteins including, but not limited to, protein ligands, soluble receptors, antibodies, antibody fragments, variable fragment single-chain antibodies (scFv), and small modular immunopharmaceutical products (SMIP™ products). Compositions and methods disclosed herein are effective in achieving the deaggregation of binding proteins while retaining a high level of functional activity.

As used in this specification and the appended claims, the singular forms “a,” “an” and “the” include plural references unless the content clearly dictates otherwise.

The practice of the present invention will employ, unless indicated specifically to the contrary, conventional methods of immunology, microbiology, molecular biology, protein chemistry, and recombinant DNA techniques within the skill of the art, many of which are described below for the purpose of illustration. Such techniques are explained fully in the literature. See, e.g., Sambrook, et al., “Molecular Cloning: A Laboratory Manual” (2nd Edition, 1989); Maniatis et al., “Molecular Cloning: A Laboratory Manual” (1982); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); “Transcription and Translation” (B. Hames & S. Higgins, eds., 1984); “Animal Cell Culture” (R. Freshney, ed., 1986); Perbal, “A Practical Guide to Molecular Cloning” (1984); Ausubel et al., “Current protocols in Molecular Biology” (New York, John Wiley and Sons, 1987); Bonifacino et al., “Current Protocols in Cell Biology” (New York, John Wiley & Sons, 1999); Coligan et al., “Current Protocols in Immunology” (New York, John Wiley & Sons, 1999); and Harlow and Lane Antibodies: a Laboratory Manual Cold Spring Harbor Laboratory (1988).

All publications, patents, and patent applications cited herein, whether supra or infra, are hereby incorporated by reference in their entirety. The present invention will be better understood through the detailed description of the specific embodiments, each of which is described in detail herein below.

Definitions

As used herein, the term “binding protein” refers generally to all classes of protein ligands, soluble receptors, antibodies, antibody fragments, variable fragment single-chain antibodies (scFv), and small modular immunopharmaceutical products (SMIP™ products). Exemplified herein are binding proteins having specific binding affinity for target proteins and other molecules, including cell-surface receptors associated with diseases such as cancer and inflammatory diseases. Within certain embodiments, binding proteins have specific binding affinity for the target proteins CD20, VEGF, Her2, EGFR, and CD37. More specifically, presented herein are SMIP™ products that specifically bind to the target proteins CD20, VEGF, Her2, EGFR, and CD37.

As used herein, the term “antibody” includes monoclonal, chimeric, humanized, and fully-human antibodies as well as biological or antigen-binding fragments and/or portions thereof. Reference herein to an “antibody” includes reference to parts, fragments, precursor forms, derivatives, variants, and genetically engineered or naturally mutated forms thereof and includes amino acid substitutions and labeling with chemicals and/or radioisotopes and the like, so long as the resulting derivative and/or variant retains at least a substantial amount of target binding specificity and/or affinity. The term “antibody” broadly includes both antibody heavy and light chains as well as all isotypes of antibodies, including IgM, IgD, IgG₁, IgG₂, IgG₃, IgG₄, IgE, IgA₁ and IgA₂, and also encompasses antigen-binding fragments thereof, including, but not limited to, Fab, F(ab′)₂, Fc, and scFv.

The term “monoclonal antibody,” as used herein, refers to an antibody obtained from a population of substantially homogeneous antibodies, i.e. the individual antibodies comprising the population are identical except for naturally-occurring mutations that do not substantially affect antibody binding specificity, affinity, and/or activity.

As used herein, the term “chimeric antibodies” refers to antibody molecules comprising heavy and light chains in which non-human antibody variable domains are operably fused to human constant domains. Chimeric antibodies generally exhibit reduced immunogenicity as compared to the parental fully-non-human antibody.

As used herein, the term “humanized antibodies” refers to antibodies comprising one or more non-human complementarity determining region (CDR), a human variable domain framework region (FR), and a human heavy chain constant domain, such as the IgG₂ heavy chain constant domain and human light chain constant domain, such as the IgKappa light chain constant domain. As used herein, the term “humanized antibody” is meant to include human antibodies (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, variable domain framework residues of the human antibody are replaced by corresponding non-human residues. Humanized antibodies may also comprise residues that are found neither in the recipient antibody nor in the imported CDR or framework sequences. Methods for humanizing non-human antibodies are well known in the art. Generally, a humanized antibody has one or more amino acid residue introduced into it from a source that is non-human. Humanization can be achieved by grafting CDRs into a human supporting FR prior to fusion with an appropriate human antibody constant domain. See, Jones et al., Nature 321:522-525 (1986); Riechmann et al., Nature 332:323-327 (1988); Verhoeyen et al., Science 239:1534-1536 (1988).

As used herein, the term “variable fragment single-chain antibody” or “scFv” refers to a covalently linked V_{H}::V_{L} heterodimer that is expressed from a gene fusion including V_{H}- and V_{L}-encoding genes linked by a peptide-encoding linker. Huston et al., Proc. Nat. Acad. Sci. USA 85(16):5879-5883 (1988). A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513, 5,132,405, and 4,946,778.

As used herein, engineered fusion proteins, termed “small modular immunopharmaceutical products” or “SMIP™ products”, are as described in co-owned US Patent Publications 2003/133939, 2003/0118592, and 2005/0136049, and co-owned International Patent Publications WO02/056910, WO2005/037989, and WO2005/017148, which are all incorporated by reference herein.

A target-specific binding protein, such as an antibody or antigen-binding fragment thereof, is said to “specifically bind,” “immunologically bind,” and/or is “immunologically reactive” to a target if it reacts at a detectable level (within, for example, an ELISA assay) with the target, and does not react detectably with unrelated polypeptides under similar conditions.

“Immunological binding,” as used in this context, generally refers to the non-covalent interactions of the type that occur between an antibody and an antigen for which the antibody is specific. The strength, or affinity of antibody-target binding interactions can be expressed in terms of the dissociation constant (K_{d}) of the interaction, wherein a smaller K_{d} represents a greater affinity. Immunological binding properties of target-specific antibodies can be quantified using methods well known in the art. One such method entails measuring the rates of target-specific antibody/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al., Annual Rev. Biochem. 59:439-473 (1990). By “specifically bind” herein is meant that the binding proteins bind to target polypeptides, proteins and/or other molecules with a dissociation constant in the range of at least 10⁻⁶-10⁻⁹ M, more commonly at least 10⁻⁷-10⁻⁹ M.

An “antigen-binding site” or “binding portion” of a target-specific antibody refers to the part of the antibody molecule that participates in target binding. The antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” or “complementarity determining regions (CDRs)” that are interposed between more conserved flanking stretches known as “framework regions,” or “FRs”.

As used herein, the term “protein aggregate” refers to the non-specific and non-native association between two or more binding proteins. Protein aggregates may include dimers, trimers, tetramers, and higher order multimers of binding proteins. The presence of protein aggregates within a pharmaceutical composition, especially pharmaceutical compositions formulated for parenteral delivery, is associated with adverse in vivo reactions including anaphylactic shock. See, e.g., Moore and Leppart, J. Clin. Enodcrin. and Metab. 51:691-697 (1980); Ratner et al., Diabetes 39:728-733 (1990); and Thornton and Ballow, Arch. Neurology 50:135-136 (1993).

As used herein, the term “biological activity” refers to both the binding protein's capacity for target-specific binding as well as capacity to mediate its native biological functionalities.

As used herein, the term “chaotrope” or “chaotropic agent” refers to compounds including, but not limited to, guanidine hydrochloride (aka, guanidinium hydrochloride, GdmHCl), sodium thiocyanate, urea, arginine, and/or a detergent. Chaotropes have in common the capacity to disrupt noncovalent intermolecular bonding between protein monomers or dimers, wherein monomers or dimers represent the native state of the binding protein.

As used herein, the term “buffer” or “buffering agent” refers to a compound or combination of compounds that is added to a composition to achieve a desired pH value or pH range. Buffers are generally classified as inorganic buffers (exemplified by phosphate and carbonate buffers) and organic buffers (exemplified by citrate, Tris, MOPS, MES, and HEPES buffers). Other buffers and buffering agents may also be employed in compositions and methods presented herein.

As used herein, the term “host cell” refers to a prokaryotic or eukaryotic cell such as a bacterial, yeast, insect, mammalian, or plant cell that is transformed or transfected such that it expresses a heterologous binding protein of interest. Exemplary host cells include, but are not limited to, Escherichia coli, Saccharomyces cerevisia, Pichia pastoris, SF9, COS, and CHO cells.

Compositions for the Deaggregation of Binding Proteins

As indicated above, the present invention provides compositions that are suitable for achieving the deaggregation of binding proteins. Compositions disclosed herein may be suitably employed for the deaggregation of solutions comprising high concentrations of binding protein, typically in the range of between about 0.1 mg/ml to about 50 mg/ml, more typically between about 0.5 and about 20 mg/ml or between about 1 mg/ml and about 10 mg/ml. Exemplified herein are binding protein solutions of about 1 mg/ml, about 2 mg/ml, about 5 mg/ml, about 8 mg/ml, and about 10 mg/ml.

Compositions of the present invention generally comprise buffer systems that are compatible with GMP manufacturing processes. For example, suitable buffer systems may include one or more salt(s) including, but not limited to, Sodium Acetate and/or Sodium Chloride. Other salts may be advantageously employed. Suitable concentration ranges for each of these salts is from about 1 mM to about 100 mM, more typically from about 5 mM to about 50 mM or from about 10 mM to about 25 mM. Exemplified herein is a buffer system comprising 25 mM Sodium Acetate and 25 mM Sodium Chloride.

The compositions for deaggregating binding proteins presented herein additionally comprise one or more chaotropic agent(s) including, but not limited to, one or more of guanidine hydrochloride, arginine, and urea. It will be understood that the precise concentration of chaotropic agent will depend upon the nature of the binding protein and its sensitivity to the chaotropic agent, but will be limited to concentrations that permit retention of biological activity of the protein in its native form. Typically, each chaotropic agent(s) is present in compositions at a concentration range from about 0.1M to about 8M. More typically, each chaotropic agent(s) is present at a concentration range from about 0.5M to about 6M, even more typically from about 1M to about 5M or from about 3M to about 5M. Exemplified herein are compositions comprising one or more chaotrope(s) at concentrations of about 3M, 3.5M, 4M, 4.5M and 5M.

Within related aspects, it will be appreciated that synergistic effects between combinations of two or more chaotropes may be advantageously achieved. For example, the present invention contemplates compositions and methods employing urea in combination with guanidine hydrochloride, urea in combination with arginine, and guanidine hydrochloride in combination with arginine. Regardless of the combination of chaotropes employed, each chaotropic agent(s) is present in compositions at a concentration range from about 0.1M to about 8M. More typically, each chaotropic agent(s) is present at a concentration range from about 0.5M to about 6M, even more typically from about 1M to about 5M or from about 3M to about 5M.

Regardless of the precise salt content and concentration, compositions provided herein are typically adjusted to a slightly acidic pH, typically in the range of about pH 4 to about pH 7, more typically in the range from about pH 5 to about pH 6. Exemplified herein are compositions buffered to about pH 5, about pH 5.5, and about pH 6. It will be understood that, as a general rule, compositions comprising higher concentrations of chaotropic agent(s) are typically buffered to a higher pH whereas compositions comprising lower concentrations of chaotropic agent(s) are typically buffered to a lower pH. Thus, for example, compositions comprising a chaotropic agent at about 3M are typically buffered to about pH 5 whereas compositions comprising a chaotropic agent at about 4M are buffered to about pH 6. Other suitable compositions comprise a chaotropic agent at about 3.5M, which are buffered to about pH 5.5. Other buffer systems may be suitably employed.

Using buffer systems as described above, high levels of deaggregation are achieved with one or more chaotrope at concentrations of between about 3M and about 4M urea over a time period of about 24 hours. As described in further detail herein, the activity of the binding protein is insensitive to protein concentration and the accumulation of high molecular weight (HMW) aggregates does not occur.

Within certain aspects of the present invention, compositions may additionally comprise one or more oxidizing agent(s) and/or one or more reducing agent(s) such as, for example, Tris(2-carboxyethyl)phosphine hydrochloride (TCEP), beta-mercaptoethanol (BME), dithiothreitol (DTT), and glutathione (GSH). It will be appreciated by those of skill in the art that addition of reducing agents is particularly advantageous for use with binding proteins wherein intra- and/or inter-molecular disulfide bonds are not required to provide stabilization of the protein's tertiary and/or quaternary structure. DTT is typically present in compositions at between about 1 mM and about 50 mM. GSH is typically present in compositions at between about 1 μM and about 100 μM, more typically between about 5 μM and about 20 μM.

Within still further aspects, compositions may additional or alternatively comprise one or more chelating agent exemplified by DTPA (Diethylenetriaminepentaacetic acid; Diethylenetriamine-N,N,N′,N′,N″-pentaacetic acid; Pentetic acid; N,N-Bis(2-(bis-(carboxymethyl)amino)ethyl)-glycine; Diethylenetriamine pentaacetic acid, [[(Carboxymethyl)imino]bis(ethylenenitrilo)]-tetra-acetic acid); EDTA (Edetic acid; Ethylenedinitrilotetraacetic acid; EDTA, free base; EDTA free acid; Ethylenediamine-N,N,N′,N′-tetraacetic acid; Hampene; Versene; N,N′-1,2-Ethane diylbis-(N-(carboxymethyl)glycine); Ethylene Diamine Tetraacetic Acid); and NTA (N,N-bis(carboxymethyl)glycine; Triglycollamic acid; Trilone A; alpha,alpha′,alpha″-trimethylaminetricarboxylic acid; Tri(carboxymethyl)amine; Aminotriacetic acid; Hampshire nta acid; nitrilo-2,2′,2″-triacetic acid; Titriplex i; Nitrilotriacetic acid). Other chelating agents may be suitably employed.

Compositions of the present invention may be suitably employed at a wide range of temperatures between the freezing point of the particular composition and the temperature at which the binding protein exhibits a substantial degree of thermal denaturation. Thus, for example, compositions and methods may be employed at between about −10° C. and about 50° C. More typically, however, compositions and methods are employed at between about −10° C. and about 37° C.; still more typically at between about 0° C. and about 30° C. or between about 10° C. and about 25° C. It will be understood, however, that the optimal temperature for a given composition and method will depend in substantial part upon the biophysical properties of the particular binding protein employed.

The deaggregation of a wide variety of binding proteins may be satisfactorily obtained with the compositions and methods presented herein. Exemplified herein are binding proteins having specific binding affinity for CD20, VEGF, Her2, EGFR, and CD37. For example, the present invention is exemplified by compositions and methods for deaggregation of a SMIP™ product having specific binding affinity for CD20.

Binding proteins deaggregated by the compositions of the present invention display substantial levels of in vitro activity as evidenced by binding and functional assays as well as substantial levels of in vivo activity. For example, the CD20 specific SMIP™ product presented herein displays substantial levels of specific binding to CD20 antigen expressed on the surface of the WIL-2S cell line as well as substantial levels of complement-dependent cytotoxicity (CDC) activity in an in vitro complement fixation assay as compared to non-treated SMIP™ product.

Methods for the Deaggregation of Binding Proteins

As indicated above, the present invention also provides methods for the deaggregation of a wide variety of binding proteins including, but not limited to, protein ligands, soluble receptors, antibodies, variable fragment single-chain antibodies (scFv), and small modular immunopharmaceutical products (SMIP™ products).

The inventive methods disclosed herein are suitably employed with high concentrations of binding proteins, as indicated above, generally in the range of about 0.1 mg/ml to about 50 mg/ml. Exemplified herein are methods for achieveing the deaggregation of binding proteins at concentrations of 5 mg/ml, 8 mg/ml, and 10 mg/ml. It will be understood, however, that the present methods may be applied to a wide variety of concentrated binding protein solutions.

In brief, a suitable cell or cell-line is selected for the expression of a binding protein of intererest and is transformed or transfected with a plasmid vector or other suitable expression system carrying a gene to be expressed. A suspension comprising a mixture of aggregated and deaggregated binding protein is isolated from the cell or culture supernatant, concentrated as appropriate, and subjected to one or more steps of protein isolation and viral inactivation. Concentrated binding protein is exchanged into a suitable buffer system, exemplified herein by a buffer system comprising 25 mM NaOAc and 25 mM NaCl. It will be understood, however, that the precise salts and concentrations may be modified in consideration of the biophysical properties of the binding protein of interest.

Typically, one or more chaotrope, such as for example guanidine hydrochloride, arginine and/or urea, is added to the buffered binding protein at a concentration of between about 2M and about 5M. More typically, the one or more chaotrope is added to the buffered binding protein at a concentration of between about 3M and about 4M. For example the one or more chaotrope may be added to the buffered binding protein at a concentration of about 3M, about 3.2M, about 3.4M, about 3.6M, about 3.8M, or about 4M.

Depending upon the precise binding protein, chaotrope and/or buffer system employed, and in consideration of the concentration of chaotrope, the solution is typically adjusted to a pH of between about pH 4 and about pH 7. More typically, the pH of the binding protein, chaotrope, buffer system solution is at a pH of between about pH 5 and about pH 6. As described above, it was determined for the exemplary binding protein disclosed herein that for solutions comprising one or more chaotropes at 3M, a pH of about pH 5 is suitable for achieving protein deaggregation. Alternatively, for solutions comprising one or more chaotropes at 4M, a pH of about pH 6 may be suitable as well. The precise combination of chaotropes and pH may depend, in part, on the biophysical properties of the binding protein in need of deaggregation, which combination may be achieved by the skilled artisan through routine experimentation in view of the guidance provided herein.

It will be futher appreciated that the present methods may further employ the addition of one or more reducing agent such as, for example, Tris(2-carboxyethyl)phosphine hydrochloride (TCEP), beta-mercaptoethanol (BME), dithiothreitol (DTT), and glutathione (GSH) and/or one or more chelating agent such as, for example, DTPA, EDTA, and/or NTA.

The present methods are suitable for the deaggregation of a wide variety of binding proteins exemplified herein by binding proteins having specific binding affinity for CD20, VEGF, Her2, EGFR, and CD37. For example, the present invention is exemplified by methods for the deaggregation of a SMIP™ product having specific binding affinity for CD20.

To achieve a high degree of binding protein deaggregation, it may be advantageous to hold the binding protein, chaotrope, buffer system solution at a temperature of between about 0° C. and about 100° C. for a period of between about 5 hours and about 24 hours. For example, in order to achieve deaggregation of the binding protein presented herein, the solution was held at a temperature of abou 25° C. (i.e. room temperature) for a period of between about 5 hours and about 20 hours.

Following the holding period, deaggregated proteins are typically exchanged into a buffer system, such as a 25 mM NaOAc, 25 mM NaCl buffer system at pH 5. Under these conditions, deaggregated proteins are stable and do not undergo substantial reaggregation. Subsequent steps of protein purification and viral filtration may, optionally, be performed in order to achieve a highly purified solution comprising the deaggregated binding protein of interest.

Assay Systems for Assessing the Biological Activity of Deaggregated Binding Proteins

Binding proteins that are deaggregated by use of the compositions and methods disclosed herein may be tested for biological activity by a number of suitable methodologies available in the art including, generally, assay systems for assessing specific binding activity and affinity as well as assay systems for assessing other functional activities. As used herein, the terms “functionally active” and “functional activity” refer to target-specific biologic and/or immunologic activities of the native, nonaggregated binding protein.

For example, the CD20 specific SMIP™ product deaggregated by the exemplary methods presented herein displays substantial levels of specific binding to CD20 antigen expressed on the surface of the WIL-2S cell line as well as substantial levels of complement-dependent cytotoxicity (CDC) activity in an in vitro complement fixation assay as compared to non-treated SMIP product.

The following assay systems for assessing functionality of deaggregated binding proteins isolated by employing the compositions and methods presented herein are provided by way of example, not limitation.

Assay Systems for Measuring Necrotic Cell Death

Necrosis is a passive process in which collapse of internal homeostasis leads to cellular dissolution involving a loss of integrity of the plasma membrane and subsequent swelling, followed by lysis of the cell. Schwartz et al., 1993. Necrotic cell death is characterized by loss of cell membrane integrity and permeability to dyes such as propidium iodide (PI) which is known by those in the art to bind to the DNA of cells undergoing primary and secondary necrosis. Vitale et al., Histochemistry 100:223-229 (1993) and Swat et al., J. Immunol. Methods 137:79-87 (1991). Necrosis may be distinguished from apoptosis in that cell membranes remain intact in the early stages of apoptosis. As a consequence dye exclusion assays using PI may be used in parallel with an assay for apoptosis, as described below in order to distinguish apoptotic from necrotic cell death. Fluorescent-activated cell sorter (FACS) based flow cytometry assays using PI allow for rapid evaluation and quantitation of the percentage of necrotic cells.

Assay Systems for Measuring Apoptotic Cell Death

Detection of programmed cell death or apoptosis may be accomplished as will be appreciated by those in the art. The percentage of cells undergoing apoptosis may be measured at various times after stimulation of apoptosis with or without administration of a binding protein deaggregated by use of the compositions and methods disclosed herein. The morphology of cells undergoing apoptotic cell death is generally characterized by a shrinking of the cell cytoplasm and nucleus and condensation and fragmentation of the chromatin. Wyllie et al., J. Pathol. 142:67-77 (1984).

Assay Systems for Measuring Target-specific Binding Affinity and Specificity

Binding proteins deaggregated by use of the compositions and methods described herein may also be tested for target-specific binding affinity and specificity and compared to the binding affinity and activity of native protein.

Binding proteins may be tested for exemplary antigen-binding affinity and/or specificity by any of the methodologies that are currently available in the art. For example, conventional cell panning, Western blotting and ELISA procedures may be employed to accomplish the step of screening for binding proteins having a particular specificity. A wide range of suitable immunoassay techniques is available as can be seen by reference to U.S. Pat. Nos. 4,016,043, 4,424,279, and 4,018,653, each of which is incorporated herein by reference.

In one type of assay, an unlabelled anti-binding protein antibody is immobilized on a solid support and the deaggregated binding protein to be tested is brought into contact with the immobilized antibody. After a suitable period of time sufficient to allow formation of a first complex, a target molecule labeled with a reporter molecule capable of producing a detectable signal is then added and incubated, allowing time sufficient for the formation of a second complex of immobilized antibody/binding protein/target molecule. Uncomplexed material is washed away, and the presence of the target molecule is determined by observation of a signal produced by the reporter molecule. The results may either be qualitative, by simple observation of the visible signal, or may be quantified by comparison with a control sample containing known amounts of native binding protein.

In a second type of assay, a target molecule to which the binding protein specifically binds is bound to a solid support. The binding processes are well known in the art and generally consist of cross-linking, covalently binding or physically adsorbing the target molecule to the solid support. The sample containing deaggregated binding protein to be tested is then added to the solid phase complex and incubated for a period of time sufficient (e.g., 2-40 minutes or overnight if more convenient) and under suitable conditions (e.g., from about room temperature to about 38° C., such as 25° C.) to allow binding of binding protein to the target molecule. Following the incubation period, the solid support is washed and dried and incubated with a binding protein-specific antibody to which a reporter molecule may be attached thereby permitting the detection of the binding of the binding protein-specific antibody to the deaggregated binding protein complexed to the immobilized target molecule.

The term “solid support” as used herein refers to, e.g., microtiter plates, membranes and beads, etc. For example, such solid supports may be made of glass, plastic (e.g., polystyrene), polysaccharides, nylon, nitrocellulose, or teflon, etc. The surface of such supports may be solid or porous and of any convenient shape. Suitable solid supports include glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay.

An alternative assay system involves immobilizing the deaggregated binding protein and exposing the immobilized binding protein to a target molecule that may or may not be labeled with a reporter molecule. As used herein, the term “reporter molecule” refers to a molecule that, by its chemical, biochemical, and/or physical nature, provides an analytically identifiable signal that allows the screening for binding proteins complexed with target molecules or with second antibodies. Detection may be either qualitative or quantitative. The most commonly used reporter molecules employed in assays of the type disclose herein are enzymes, fluorophores, radioisotopes, and/or chemiluminescent molecules.

In the case of an enzyme immunoassay (EIA), an enzyme is conjugated to the detection antibody or target molecule, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase. In general, the enzyme-labeled antibody is added to a potential complex between a target molecule and a binding protein, allowed to bind, and then washed to remove the excess reagent. A solution containing the appropriate substrate is then added to the complex of target antigen/deaggregated binding protein/labeled-antibody. The substrate reacts with the enzyme linked to the labeled antibody, giving a qualitative visual signal, which may be further quantified, usually spectrophotometrically, to indicate the activity of the deaggregated binding protein present in the sample.

Alternatively, fluorescent compounds, such as fluorescein and rhodamine, or fluorescent proteins such as phycoerythrin, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic color visually detectable with a light microscope or other optical instruments. As in the EIA, the fluorescent labeled antibody is allowed to bind to the antigen-antibody complex. After removing unbound reagent, the remaining tertiary complex is exposed to light of the appropriate wavelength. The fluorescence observed indicates the presence of the bound binding protein of interest.

Immunofluorescence and EIA techniques are both well established in the art. It will be understood that other reporter molecules, such as radioisotopes, and chemiluminescent and/or bioluminescent molecules, may also be suitably employed in the screening methods disclosed herein.

The following examples serve to more fully describe the manner of using the above-described invention, as well as to set forth the best modes contemplated for carrying out various aspects of the invention. These examples in no way serve to limit the true scope of this invention, but rather are presented for illustrative purposes.

### EXAMPLES

### Example 1

### Size-Exclusion High Performance Liquid Chromatography (SEC-HPLC) System for the Separation of Protein Aggregates and Non-Aggregated Protein

This Example demonstrates a size-exclusion high performance chromatography (SEC-HPLC) system for separating multimeric protein aggregates from active non-aggregated protein-of-interest (POI) for an exemplary CD20-specific SMIP™ product.

Samples of a CD20-specific SMIP™ product were analyzed by size-exclusion high performance liquid chromatography (SEC-HPLC; Progel-TSK G3000 SWXL HPLC column; Tosoh Bioscience LLC, Montgomeryville, Pa.) and peak areas integrated. See Table 1 for SEC-HPLC operating parameters.

TABLE 1
SEC-HPLC Operating Parameters

| | |
|---|---|
| Column Size | 7.8 mm (ID) × 30.0 cm (L) |
| Pore Size | 5 μm |
| Maximum Pressure of the | 70 kg/cm² |
| Column | |
| Operating Temperature | Ambient |
| Sample Temperature | Ambient |
| Detection/Reference | 280 nm/n/a with Shimadzu |
| Wavelength | equipment |
| Bandwidth (nm) | 2 nm |
| Mobile Phase | dPBS + 0.05% Sodium Azide |
| Flow Rate | 1 ml/min |
| Run Length | 15 minutes |
| Amount of Protein Sample | 250 μg |
| Injected | |
| Integration Method | Perpendicular Drop |

The linear range for the bulk product of the CD20-specific SMIP™ product using the TSK G3000SWXL column was determined as 10 μg to 500 μg (r²≧0.99), with acceptable repeatability of the relative purity across the range 50 μg to 500 μg (sd≦0.1%). Interactions between product and column matrix were observed resulting in an underestimation of the molecular weight for the CD20-specific SMIP™ product POI (81 kDa compared to a theoretical molecular weight of 107 kDa for the dimeric CD20-specific SMIP™ product and slight peak tailing of the protein of interest.

Visual inspection of the linearity plots showed that the peak area response was linear up to a load of 500 μg (data not shown). A summary of the results generated by regression analysis is shown in Table 2. Regression analysis performed on the POI peak area data for loads of 9 μg to 491 μg gave a square of the correlation coefficient (r²) of 0.997 and an intercept value of 41.65 peak area units. The response across the load range of 9 μg to 491 μg gave acceptable linearity (r²≧0.99).

TABLE 2
Regression Analysis of Load 9 μg to 491 μg and
Peak Area for a CD20-specific SMIP ™ product
130 L Bulk Product ‘Protein of Interest’

| Parameter | Value |
|---|---|
| Intercept (mAU*s) | 41.6476 |
| Slope (mAU*s/μg) | 95.3907 |
| r² | 0.9974 |

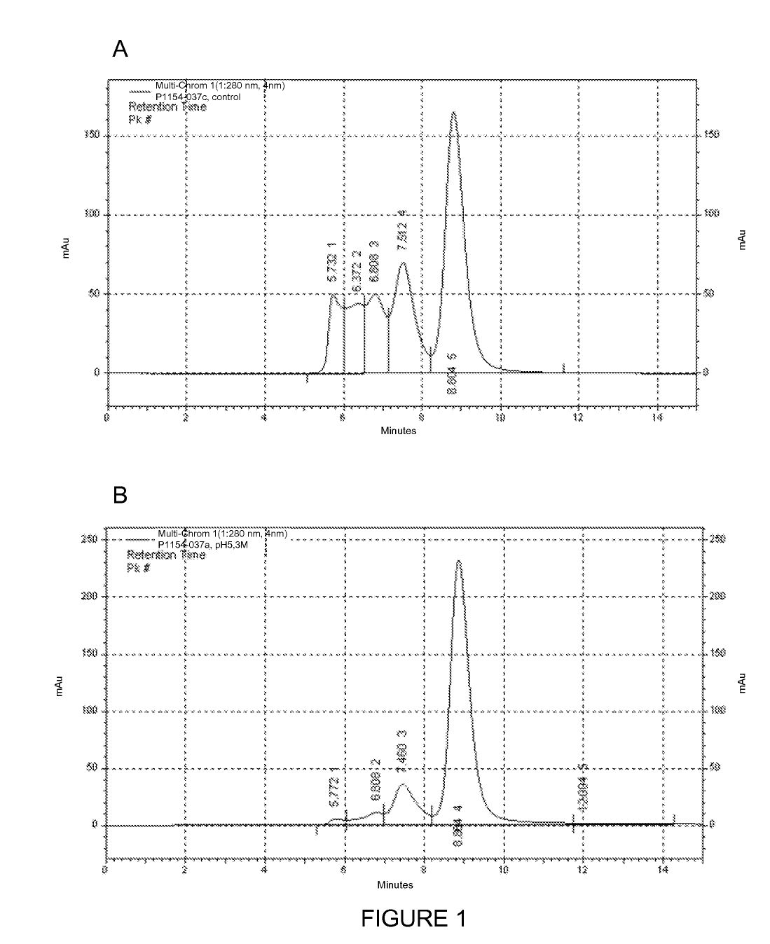

FIGS. 1A and 1B present chromatographic traces showing the time-dependent elution of protein aggregates and POI for an exemplary CD20-specific SMIP™ product from a Protein A chromatography column eluted with a single step of Protein A at pH 5. The data presented in FIG. 1A were obtained with a binding protein applied to the column in a control buffer comprising 25 mM NaCl, 25 mM NaOAc at pH 5. The data presented in FIB. 1B were obtained with the same binding protein applied to the column following a 20-hour treatment with a solution comprising 25 mM NaCl, 25 mM NaOAc, 3M urea at pH 5. The % POI obtained in FIG. 1A was 46.8% (see Table 3) whereas the % POI obtained in FIG. 1B was 80.1% (see Table 4).

TABLE 3
SEC-HPLC Separation of CD20-specific SMIP ™ Product
Aggregates from Non-aggregated CD20-specific SMIP ™ Product
in 25 mM NaCl/25 mM NaOAc, pH 5

| | | | Percent Total | |
|---|---|---|---|---|
| Peak No. | Retention Time | Peak Area | Peak Area | Peak Height |
| 1 | 5.732 | 1187786 | 9.284 | 50362 |
| 2 | 6.372 | 1295781 | 10.128 | 44255 |
| 3 | 6.808 | 1603612 | 12.534 | 50174 |
| 4 | 7.512 | 2718219 | 21.247 | 70023 |
| 5 (POI) | 8.804 | 5988243 | 46.806 | 165024 |
| Totals | | 12793641 | 100.000 | 379838 |

TABLE 4
SEC-HPLC Separation of CD20-specific SMIP ™ product
Aggregates from Non-aggregated CD20-specific SMIP ™ product
in 25 mM NaCl/25 mM NaOAc, 3 M Urea, pH 5

| | | | Percent Total | |
|---|---|---|---|---|
| Peak No. | Retention Time | Peak Area | Peak Area | Peak Height |
| 1 | 5.772 | 130273 | 1.252 | 5174 |
| 2 | 6.808 | 416329 | 4.002 | 10699 |
| 3 | 7.460 | 1465905 | 14.092 | 35220 |
| 4 (POI) | 8.864 | 8337188 | 80.149 | 231667 |
| 5 | 12.004 | 52432 | 0.504 | 675 |
| Totals | | 10402127 | 100.000 | 283435 |

### Example 2

### Composition-Dependent Increase in Deaggregation for a CD20-Specific SMTP™ Product

This example demonstrates that compositions comprising various concentrations of chaotrope in acidic buffer solutions comprising NaOAc and NaCl are effective in increasing the yield of active, deaggregated protein-of-interest (POI) for an exemplary CD20-specific SMIP™ product.

In a first experiment, a 10 ml sample of a 16.3 mg/ml Protein A eluate of a CD20-specific SMIP product was dialysed overnight against 1 liter of phosphate buffered saline (PBS), pH 7.0. In parallel, a second 10 ml sample of the same 16.3 mg/ml Protein A eluate of the exemplary CD20-specific SMIP™ product was dialyzed overnight against 1 liter of 25 mM NaOAc/25 mM NaCl, pH 5.0.

Each sample was removed from dialysis, diluted to 5 or 10 mg/ml in the respective dialysis buffer, and adjusted to a final urea concentration of 0M, 3M, or 4M for a total of 12 samples. These samples were incubated at room temperature for 22 hours.

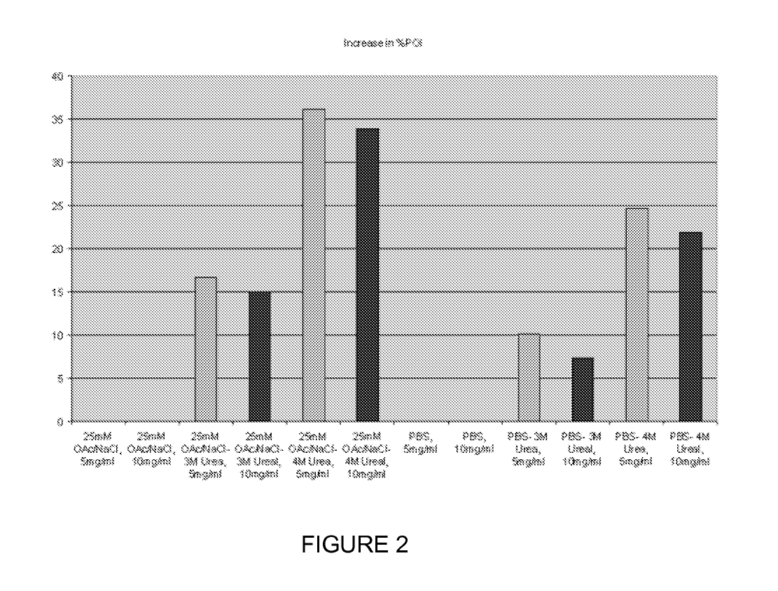

10 μl of each of the 12 samples were analyzed by size-exclusion high performance liquid chromatography (as described in Example 1) and peak areas were integrated. The relative peak area of the 0M urea control Peak of Interest (POI) at retention time ˜8.8 minutes was set at 100%, and relative increase in the areas of the experimental group POI was charted for each sample. See FIG. 2.

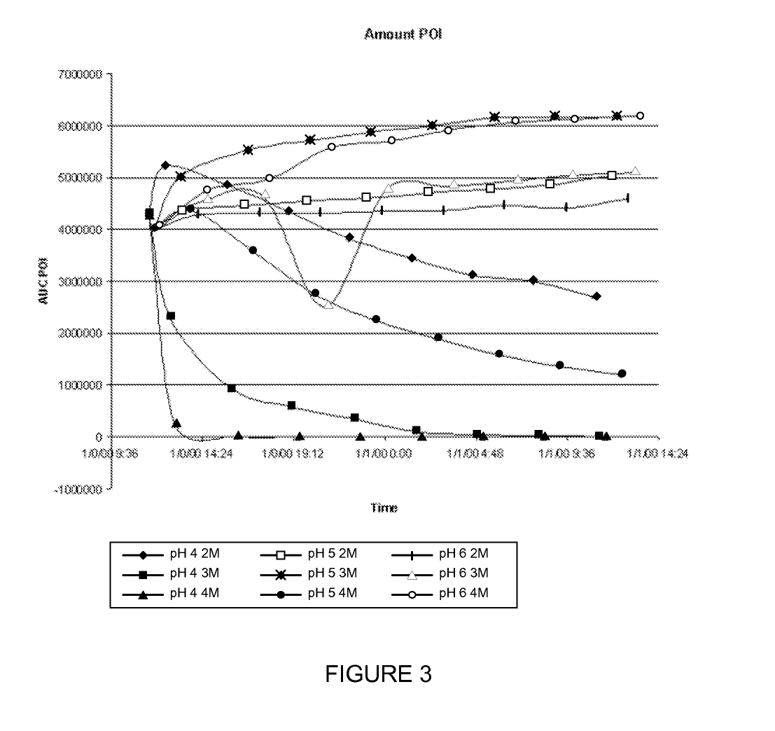

In a second experiment, the time course for deaggregation of an exemplary CD20-specific SMIP™ product was determined at pH 4.0, pH 5.0, and pH 6.0 with 3M and 4M urea. 8 ml of CD20-specific SMIP™ product Protein A eluate at 16.3 mg/ml was dialyzed overnight against 500 ml of 25 mM NaOAc/25 mM NaCl at pH 4.0, 5.0, or 6.0. The three samples were diluted with the respective dialysis buffer and urea to a final concentration of 8 mg/ml CD20-specific SMIP™ product and 0M, 2M, 3M or 4M urea. The 0M urea samples were analyzed by SEC HPLC and the POI peak areas were set to be the t=0 time point. 12 μl of the 2M, 3M, and 4M urea concentration samples at pH 4.0, 5.0, and 6.0 were injected and analyzed sequentially by SEC HPLC, with the entire sequence repeated over the course of ˜24 hours. The total peak area of the POI was plotted against the injection time to create a time course of deaggregation at these 9 conditions. The results of this experiment are summarized in FIG. 3.

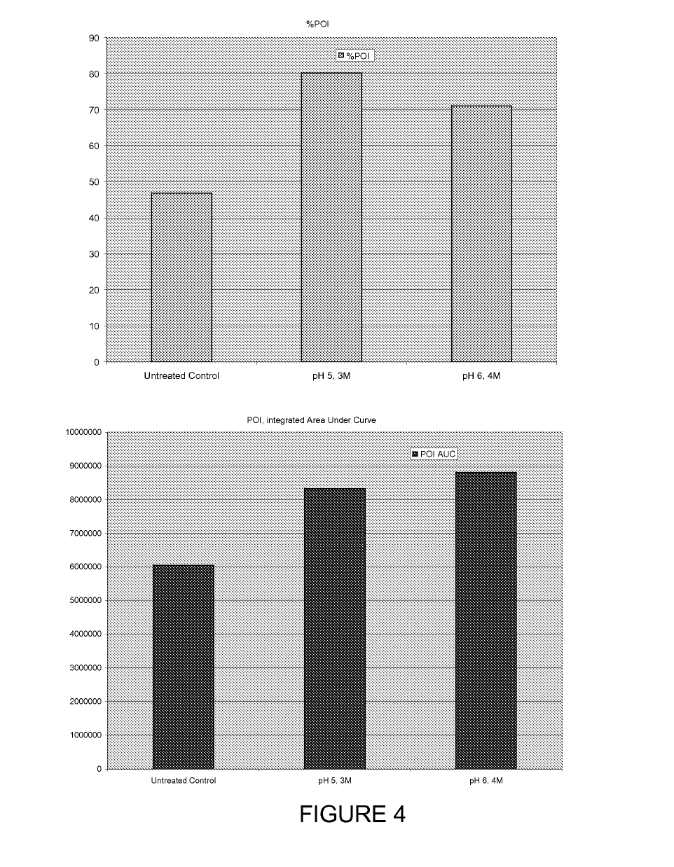

In a third experiment, the urea-dependent deaggregation of an exemplary CD20-specific SMIP™ product was measured at pH 5, 3 M urea and at pH 6, 4 M urea. Two 2 ml samples of the exemplary CD20-specific SMIP™ product Protein A eluate at 16.3 mg/ml were dialyzed overnight against 300 ml of 25 mM NaOAc/25 mM NaCl at pH 5.0 or pH 6.0, respectively. The pH 5.0 sample was adjusted to 8 mg/ml CD20-specific SMIP™ product and 3 M urea in buffer containing 25 mM NaOAc/25 mM NaCl at pH 5.0. The pH 6.0 sample was adjusted to 8 mg/ml CD20-specific SMIP™ product and 4 M urea in buffer containing 25 mM NaOAc/25 mM NaCl at pH 6.0. These samples were incubated at room temperature for 20 hours. Both samples were exchanged into PBS by 5 hr dialysis. Both samples were analyzed by SEC HPLC and the total POI areas and % POI of total were plotted by bar graph. See FIG. 4.

### Example 3

### In vitro Characterization of a Deaggregated CD20-Specific SMIP™ Product

This Example demonstrates the in vitro activity of a CD20-specific SMIP™ product deaggregated by the compositions and methods of the present invention.

The cytotoxic effect of an exemplary CD20-specific SMIP™ product, in combination with complement, on cancer cells is measured based on the cellular metabolic reduction of AlamarBlue™ dye. A human B-lymphoblastoid cell line, WIL2-S, is used in combination with an exemplary CD20-specific SMIP™ product and rabbit complement in a 96-well format. The appropriate controls and product sample concentrations are added and allowed to incubate at 37° C., 5% CO₂. The AlamarBlue™ dye solution is then added. The dye is reduced by cellular metabolism into a form that is read fluorometrically at a set time point. The relative fluorescence units (RFUs) are directly proportional to the viable cell number in each sample.

The target affinity of the exemplary CD20-specific SMIP™ product on a CD20 expressing cell line is measured based on the relative fluorescence of a fluoresecin isothiocyanate (FITC) conjugated stain that binds to the CD20-specific SMIP™ product in a dose dependent manner. A human B-lymphoblastoid cell line, WIL2-S, is incubated with various dilutions of the CD20-specific SMIP™ product, allowing it to bind the cellular target. The cells are washed to remove any unbound CD20-specific SMIP™ product and stained for detection of the bound protein. The cells are washed to remove any unbound stain and analyzed by flow cytometery (FACS) for FITC geometric mean fluorescence intensity (GMFI). Data are fit to 4-parameter curves and the ED50 values calculated. Results are reported as % Relative Potency (sample vs. reference standard).

### Example 4

In Vivo Characterization of a Deaggregated CD20-Specific SMIP™ Product

This Example discloses a Ramos tumor cell animal model system for assessing the in vivo activity of a CD20-specific SMIP™ product, deaggregated by the compositions and methods of the present invention.

Ramos cells are cultured to appropriate confluency and >90% viability, harvested, and washed 2× with sterile PBS. Harvested cells are resuspended to an appropriate cell number for injection (i.e. 100 μl/mouse; for 5×10⁶ cells/mouse, cells are resuspended to 5×10⁷ cells/ml) and held on ice until injection. Using a 27 G ½ in. needle, 100 μl of cell suspension is injected subcutaneously on the right flank of the mouse, which typically yields a visible blister. Mice are observed daily for tumor growth. Tumors are typically established when they reach ˜150-300 mm³.

On day 0, animals are sorted and grouped according to tumor size (using LabCat software; Innovative Programming Associates, Inc., Princeton, N.J.) and body weights are recorded. Tumors are measured 2-3× weekly and body weights monitored weekly. Animals are maintained until tumors reach no larger than 1500 mm³. Animals are sacrificed if ulceration of tumor occurs, if there is an extreme loss in body weight, if the tumor exceeds 1500 mm³, and/or if the tumor inhibits an animal's mobility. Studies are typically terminated after day 90.

We claim:

1. A composition for the deaggregation of a binding protein, said composition comprising a salt at a concentration of between about 1 mM and about 100 mM and a chaotropic agent at a concentration of between about 0.1M and about 8M, wherein said composition has a pH of between about pH 4 and about pH 7.

2. The composition of claim 1 wherein said salt is at a concentration of between about 10 mM and about 25 mM.

3. The composition of claim 1 wherein said salt is selected from the group consisting of NaCl and NaOAc.

4. The composition of claim 1 wherein said chaotropic agent is at a concentration of between about 3M and about 5M.

5. The composition of claim 1 wherein said chaotropic agent is selected from the group consisting of guanidine, arginine, and urea.

6. The composition of claim 1 wherein said composition has a pH of between about pH 5 and about pH 6.

7. The composition of claim 1, further comprising a reducing agent selected from the group consisting of Tris(2-carboxyethyl)phosphine hydrochloride (TCEP), beta-mercaptoethanol (BME), dithiothreitol (DTT), and glutathione (GSH).

8. The composition of claim 1, further comprising a chelating agent selected from the group consisting of DTPA, EDTA, and NTA.

9. A method for the deaggregation of a binding protein, said method comprising the steps of:
(a) suspending a mixture comprising a non-aggregated binding protein and an aggregated binding protein to a concentration of between about 0.1 mg/ml and about 50 mg/ml in a composition comprising a salt at a concentration of between about 1 mM and about 100 mM and a chaotropic agent at a concentration of between about 0.1M and about 8M, thereby achieving a binding protein suspension;
(b) adjusting the pH of said binding protein suspension to a pH of between about pH 4 and about pH 7; and
(c) holding said binding protein suspension at a temperature of between about −10° C. and about 50° C. for between about 5 hours and about 24 hours,
thereby increasing the percentage of non-aggregated binding protein and decreasing the percentage of aggregated binding protein.

10. The method of claim 9, further comprising the step of exchanging said binding protein suspension into a buffer system comprising a salt, wherein said buffer system is at a pH of about pH 5.

11. The method of claim 10, further comprising the step of separating said non-aggregated binding protein from said aggregated binding protein.

12. The method of claim 9 wherein said binding protein is selected from the group consisting of a protein ligand, a soluble receptor, an antibody, an antibody fragment, a variable fragment single-chain antibody (scFv), and a small modular immunopharmaceutical product.

13. The method of claim 12 wherein said binding protein is suspended to a concentration of between about 1 mg/ml and about 50 mg/ml.

14. The method of claim 12 wherein said salt concentration is between about 10 mM and about 25 mM.

15. The method of claim 12 wherein said salt is selected from the group consisting of NaOAc and NaCl.

16. The method of claim 12 wherein said chaotropic agent is at a concentration of between about 3M and about 5M.

17. The method of claim 12 wherein said chaotropic agent is selected from the group consisting of guanidine, arginine, and urea.

18. The method of claim 12 wherein said binding protein suspension is adjusted to a pH of between about pH 5 and about pH 6.

19. The method of claim 12 wherein said binding protein has specific binding affinity for a target protein selected from the group consisting of CD20, VEGF, Her2, EGFR, and CD37.

20. The method of claim 19 wherein said binding protein is a small modular immunopharmaceutical product wherein said small modular immunopharmaceutical product binds to said target protein with a dissociation constant in the range of at least 10⁻⁶-10⁻⁹ M.
